(19) United States
(12) Patent Application Publication
(10) Pub. No.: US 2020/0100842 A1
(43) Pub. Date: Apr. 2, 2020

(54) SYSTEMS AND METHODS FOR IMAGE-GUIDED MEDICAL PROCEDURES

(71) Applicant: Wake Forest University Health Sciences, Winston-Salem, NC (US)

(72) Inventors: Scotty A. Chung, Winston-Salem, NC (US); Philip J. Brown, Winston-Salem, NC (US)

(21) Appl. No.: 16/591,213

(22) Filed: Oct. 2, 2019

Related U.S. Application Data
(60) Provisional application No. 62/739,959, filed on Oct. 2, 2018.

(51) Int. Cl. A61B 34/10; A61B 34/20; A61B 6/00; A61B 90/00

(52) U.S. Cl. CPC A61B 34/10; A61B 34/20; A61B 6/52; A61B 90/36; A61B 2034/105; A61B 2090/374; A61B 2090/3762; A61B 2034/102; A61B 2034/2055

(57) ABSTRACT

The present disclosure relates to systems and methods for carrying out image-guided medical procedures. In some aspects and embodiments, the disclosure provides systems and methods for improving the accuracy of such procedures, especially where multiple tools are used to carry out the procedure.

### CROSS-REFERENCE TO RELATED APPLICATIONS

This applications claims the benefit of U.S. Provisional Application No. 62/739,959 filed Oct. 2, 2018, which is hereby incorporated by reference in its entirety.

### TECHNICAL FIELD

The present disclosure relates to systems and methods for carrying out image-guided medical procedures. In some aspects and embodiments, the disclosure provides systems and methods for improving the accuracy of such procedures, especially where multiple tools are used to carry out the procedure.

### DESCRIPTION OF RELATED ART

Surgery and other medical procedures are increasingly carried out with the guidance of imaging technology. Such techniques allow a medical professional access internal locations within the body of a patient in ways that are minimally invasive. The imaging technology allows the medical professional to observe internal locations in ways that once would have required invasive procedures and the removal of some amount of visceral tissue. In most instances, such image-guided techniques require the imaging of a certain portion of the patient's body, and the display of that image on a monitor or other display device.

But this presents certain challenges. Surgery and various medical procedures occur in three-dimensional space, but such imaging techniques may only provide two-dimensional information, or may provide three-dimensional information poorly. Moreover, certain medical professionals may experience difficulty coordinating hand and finger motions to what they observe on the screen.

Therefore, there is a continuing need to develop novel apparatuses and methods for improving the accuracy and utility of image-guided medical procedures.

### SUMMARY

The present disclosure provides apparatuses and methods that overcome one or more of the problems described above. In certain embodiments, the present disclosure provides apparatuses and methods that enhance the video display by overlaying a feature that shows the projected path of a surgical instrument or other device, so that users can recognize whether the projected path lies over an intended target region in the interior of a subject. These features can be incorporated into a stand-alone device, or function an external add-on.

In a first aspect, the disclosure provides apparatuses for medical imaging, the apparatuses comprising: (a) an imaging device, which comprises a radiation source and a detector; (b) a processor, which is in electrical communication with the detector, and which is configured to receive an image data stream from the detector; (i) wherein the processor is configured to convert the image data stream to a video data stream, which provides a video representation of the image data stream; and (ii) wherein the processor is further configured to detect one or more straight lines in the image data stream, and to incorporate into the video data stream a video representation of a linear extension of the one or more straight lines in the image data stream; and (c) a video display, which is in electrical communication with the processor, and which is configured to display a video image based on the video data stream.

In a second aspect, the disclosure provides methods for imaging, the methods comprising: (a) providing an imaging device; (b) detecting an image using the imagine device, wherein the image is represented by an image data stream; (c) communicating the image data stream to a processor, and, using the processor, (i) converting the image data stream to a video data stream, which provides a video representation of the image, and (ii) detecting one or more straight lines in the image data stream, and incorporating into the video data stream a video representation of a linear extension of the one or more straight lines in the image data stream; and (d) communicating the video data stream to a video display to display a video image based on the video data stream.

In a third aspect, the disclosure provides apparatuses for enhancing a video data steam, the apparatuses comprising: (a) a video input, which is configured to receive a first video data stream; (b) a processor, which is in electrical communication with the video input, wherein the processor is configured to detect one or more straight lines in the first video data stream, and to convert the first video data stream to a second video data stream, wherein the second video stream comprises a video representation of a linear extension of the one or more straight lines in the first video data stream; and (c) a video display, which is in electrical communication with the processor, and which is configured to display a video image based on the second video data stream.

In a fourth aspect, the disclosure provides methods of enhancing a video data stream, the methods comprising: (a) providing a first video data stream, which represents a video image; (b) communicating the first video data stream to a processor, and, using the processor, detecting one or more straight lines in the image, and converting the first video data stream to a second video data stream, wherein the second video data stream comprises a video representation of a linear extension of the one or more straight lines in the image; and (c) communicating the second video data stream to a video display to display a video image based on the second video data stream.

Further aspects and embodiments are provided in the drawings, the detailed description, the claims, and the abstract.

### BRIEF DESCRIPTION OF DRAWINGS

The following drawings are provided for purposes of illustrating various embodiments of the compounds, compositions, methods, and uses disclosed herein. The drawings are provided for illustrative purposes only, and are not intended to describe any preferred compounds or compositions or any preferred methods or uses, or to serve as a source of any limitations on the scope of the claimed inventions.

FIG. 1 shows a non-limiting example of an integrated video enhancement apparatus according to certain embodiments disclosed herein.

FIG. 2 shows a non-limiting example of a process flow for a method of integrated video enhancement according to certain embodiments disclosed herein.

FIG. 3 shows a non-limiting example of a non-integrated video enhancement apparatus according to certain embodiments disclosed herein.

FIG. 4 shows a non-limiting example of a process flow for a method of non-integrated video enhancement according to certain embodiments disclosed herein.

FIG. 5 shows a non-limiting example of a final user display image according to certain embodiments disclosed herein.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, the term “user” refers to an animal. In some embodiments, the user is a mammal. In some such embodiments, the user is a human.

As used herein, “optionally” means that the subsequently described event(s) may or may not occur. In some embodiments, the optional event does not occur. In some other embodiments, the optional event does occur one or more times.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present. As used herein, the phrases “consist essentially of,” “consists essentially of,” and “consisting essentially of” refer to groups that are open, but which only includes additional unnamed members that would not materially affect the basic characteristics of the claimed subject matter.

As used herein, “or” is to be given its broadest reasonable interpretation, and is not to be limited to an either/or type of construction. Thus, the phrase “comprising A or B” means that A is present and not B, that B is present and not A, or that A and B are both present. Further, if A, for example, defines a class that can have multiple members, e.g., A₁ and A₂, then one or more members of the class can be present concurrently.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Other terms are defined in other portions of this description, even though not included in this subsection.

Integrated Video-Enhanced Imaging Apparatus

In at least one aspect, the disclosure provides apparatuses for medical imaging, the apparatuses comprising: (a) an imaging device, which comprises a radiation source and a detector; (b) a processor, which is in electrical communication with the detector, and which is configured to receive an image data stream from the detector; (i) wherein the processor is configured to convert the image data stream to a video data stream, which provides a video representation of the image data stream; and (ii) wherein the processor is further configured to detect one or more straight lines in the image data stream, and to incorporate into the video data stream a video representation of a linear extension of the one or more straight lines in the image data stream; and (c) a video display, which is in electrical communication with the processor, and which is configured to display a video image based on the video data stream.

The apparatuses disclosed herein can incorporate any suitable medical imaging device, so long the device at least includes a radiation source and a detector. Examples of such devices include, but are not limited to, fluoroscopes, CT scanners, ultrasound devices, magnetic resonance imaging (MRI) devices, x-ray devices, endoscopes, elastographs, thermographs, positron emission tomography (PET) devices, and single-photon emission computed tomography (SPECT) devices. In some embodiments, the imagine device is a fluoroscope. In some further embodiments, the fluoroscope includes an x-ray source, a filter, and a detector.

The apparatuses disclosed herein can include imaging devices having two or more radiation sources or two or more detectors. For example, in certain embodiments, the apparatus could include a fluoroscopy setup having two x-ray sources and two detectors, where each source-detector pair is positioned in different locations. This allows the user to obtain an image from multiple angles.

In apparatuses disclosed herein, the detector of the imaging device is in electrical communication with a processor. In general, the processor is configured to receive an image data stream from the detector. The nature of the image data stream may depend, to some extent, on the type of detector and whether the detector itself carries out some cursory processing of the image data. In some embodiments, the image data stream is in an analog form. In some other embodiments, the image data stream is in a digital form, e.g., when the detector itself contains a processor that converts the detected signal to a digital format.

The processor can take on any suitable form. In general, the processor includes one or more integrated circuits. In embodiments where the image data stream is in an analog form, the processor includes a video capture card, which is an integrated circuit that, among other things, includes a video decoder that converts the analog image data into a standard digital video format. In embodiments where the image data stream is already in a digital format, then, in some such embodiments, the processor includes an integrated circuit that converts the image data into a standard digital video format. Note that the phrase “configured to receive an image data stream from the detector” means that the processor is in electrical communication with the detector and includes some component capable of processing the image data stream, regardless of its format. Moreover, the phrase “configured to convert the image data stream to a video data stream, which provides a video representation of the image data stream,” refers to the various types of processing described in above, where the image data stream is converted to a standard digital video format.

As described above, the processor is also configured to detect one or more straight lines in the image data stream, and to incorporate into the video data stream a video representation of a linear extension of the one or more straight lines in the image data stream. This processing feature can be incorporated into the apparatus in any suitable way. In some embodiments, it can be encoded into an electronic circuit, which can be included on the same integrated circuit with the video decoder, or on a separate integrated circuit within the apparatus. In some other embodiments, this processing feature can be carried out by a computer that is programmed to carry out this function. Thus, in some embodiments, the processor can be an integrated circuit or a plurality of integrated circuits integrated into the apparatus. Or, in other embodiments, may be incorporated into a computer that is programmed to carry out one or more of the above processing functions.

The apparatuses disclosed herein are configured or programmed to detect one or more straight lines in the image data stream. This does not necessarily mean that the processing involved in this detection is carried out directly on the image data stream. In some embodiments, for example, the processor may convert analog image data to a standard digital video format, and then perform the detecting on the data in that standard digital video format.

These one or more straight lines generally represent or correspond to one or more surgical instruments. Examples of such surgical instruments include, but are not limited to, wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof. In that way, the processor is detecting the presence of one or more such surgical instruments within the image that is captured by the detector. This would commonly occur within the context of image-guided surgery or other image-guided medical procedures.

The detection of the one or more straight lines can be carried out by any suitable algorithm. For example, the detection can be carried out using algorithms based on the Hough transform, and tailored to identify particular features characteristic of the relevant instruments. Examples of these techniques are illustrated in literature relating to the grayscale Hough transform (GSHT), such as Lo et al., Pattern Recognition, vol. 28, pp. 647-661 (1994), which is incorporated herein by reference. The manipulation of the original image is typically required before a line detection algorithm is applied. This manipulation may include converting a color image to a grayscale image, isolating a specific region of interest, smoothing to remove edges caused by artifacts, converting to a binary image, reducing the width of large objects. Once the image has been manipulated, the Hough transform implements a voting scheme for determining the presence and location of a line within the image. Possible lines must receive a user specified number of votes to be considered detected in the image. In some embodiments, the pixels that are considered important by the binary filter each vote for the set of lines they could lie on. After iterating through all the pixels, a threshold must be reached to be considered a line.

The apparatuses disclosed herein are also configured to incorporate into the video data stream a video representation of a linear extension of the one or more straight lines in the image data stream. In some embodiments, this occurs following the above-described straight-line detection. Determining the tip or end of the device can be used to determine the direction that the linear extension should be performed. The linear extension is drawn along the detected line from the tip of the device to the edge of the image in the direction moving away from the device. The Bresenham's line algorithm is used for generating the path of the linear guide extension given two points and user defined thickness. This generated linear guide extension is then merged with the original image which may completely or partially cover the portions of the original image with the linear guide. For example, in some cases, there is a user defined transparency of combining these two images together. Thus, a user can see both what is underneath the original image and the generated guidance.

In some embodiments, the apparatuses disclosed herein include a processor that is further configured to determine a direction of motion of any of the one or more straight lines in the image data stream. To recognize the same line in a series of images, an estimation of a line's movement is required. This can be achieved by an assumption if a line in the previous image and a line in the current image have similar position and orientation, they are the same line. The speed of movement of this line can be determined and predicted for the proceeding images.

In some further embodiments, the apparatuses disclosed herein include a feature where the video data stream further comprises a video representation of an error bar. In some embodiments, the error bar corresponds to a confidence interval in calculating the linear extension of the one or more straight lines in the image data stream. In some embodiments, the confidence interval is a 80% confidence interval, or a 90% confidence interval, or a 95% confidence interval, or a 99% confidence interval. The confidence interval is a calculated value representative of the deviation from the predicted location and orientation of a line to the line detected in the current image. The calculated value can be dependent on the speed of the line to achieve an accurate confidence value for slower and faster moving line.

In some embodiments, the video data stream further comprises a video representation of a number, wherein the number is a text representation of the number of straight lines detected in the image data stream. The system can count the number of lines detected. This number is then overlaid onto the user display image as text. This text can be generated using one of the many font libraries available which, given the text content and location, will overlay the text onto the user display image.

In some embodiments, the video data stream further comprises a video representation of an indicator, wherein the indicator appears on the final user display image at each point two or more of the detected straight lines intersect. Line-line intersections in a plane can be determined using the line equation. When lines are detected, line equations are obtained. From these equations, the intersection points of all detected lines can be calculated based on Euclidian geometry. At the location of each intersection point, a font library can be used to generate a symbol that is then overlaid onto the user display image as an intersection indicator.

In some embodiments, the processor is further configured to determine that bone material is near a detected line. When the system determines that bone material, both cortical and trabecular, is near a detected line, the system will overlay the bone material region with a transparent color or outline. An embodiment of the system may use some or all of the available information to determine the presence and location of bone within the image: intensity of the pixels, the general shape of pixels, the relative location of pixels, and input generated by the user. Having determined the area of bone material within an image, the perimeter of this area can be divided into specific points. Given each point of the perimeter and each point of the previously detected line, the distance can be calculated. If the distance is shorter than a user specified amount, the bone region is then marked to be highlighted. The portion of the bone to be highlighted can be merged with a given color to produce a transparent image that is then shown on the user display image.

In some embodiments, the processor is further configured to determine a centroid of a region of detected bone, wherein all portions of the region of detected bone are weighed equally in the determination. Determining the centroid of a given region of bone can be performed by calculations of image intensity moments. For the bone area, the intensity is assumed to be constant throughout the entire region. From these moment values, the centroid is at the intersection of two neutral axis which can be calculated from the first moment. In some embodiments, the video data stream further comprises an indicator at each centroid of a region of detected bone. At each determined centroid, a font library can be used to generate a symbol that is then overlaid onto the user display image as a centroid indicator.

In some embodiments, the processor is further configured to determine a centroid of a region of detected bone, wherein portions of the region of detected bone are weighed based on their intensity in the user data stream in the determination. Determining the centroid of a given region of bone can be performed by calculations of image intensity moments. Due to the non-uniformity of bone density, the centroid calculations can use the intensity of the original image to correlate with density. From these moment values, the centroid is at the intersection of two neutral axis which can be calculated from the first moment. In some embodiments, the video data stream further comprises an indicator at each centroid of a region of detected bone. At each determined centroid, a font library can be used to generate a symbol that is then overlaid onto the user display image as a centroid indicator.

In some embodiments, the video data stream further comprises a video representation identifying at least one individual line, wherein the at least one line is identified by a unique label on the final user display image. The same line can be identified with a specific text label or color throughout a series of continuous images. Forming an estimation of the line's motion can enable the prediction of a line in the consecutive image. If a line is within a tolerance range of this predicted line, then it will be considered the same line. The same color or label for this line can be applied to the original image so said unique identifier appears on the user display image. In some embodiments, the processor detected and indicated placed screw trajectories using labels, colors, or other unique indicators. Using the same instrument detection, trajectory projection, and bone detection algorithms described previously, screws may be individually identified and labeled. Their cross section may be overlaid with a transparent color or outline using the following: intensity of the pixels, general shape of pixels, relative location of pixels, and user-generated input. The screw trajectory can be displayed as a short line segment or as a continuous line that runs through the entire user display image. This line may be used in the calculation of intersections and angles with other trajectory paths.

In some embodiments, once the processor has identified line representations from at least a pair of devices, the angle between the two lines can be calculated and displayed on the final user display image. The location of this angle may or may not be placed near the point of intersection. Additionally, a curved line may be drawn connecting the two intersecting lines. The region bordered by the two lines and the curved line may be shaded. The line equations can be determined from the line detection. The equation for two lines can then be used to directly find the angle between the two lines based on Euclidian geometry. The angle value can be placed near the intersection point using a text label from a standard font library.

The apparatuses disclosed herein also include a video display, which is in electrical communication with the processor, and which is configured to display a video image based on the video data stream. Any suitable video display can be used, including, but not limited to, a monitor, a computer screen, a television screen, and the like. The video data stream can be communicated to the video display in any suitable way, including wired or wireless transmission. In some embodiments, a standard cable having a VGA, DVI, HDMI, or DisplayPort connector can be used.

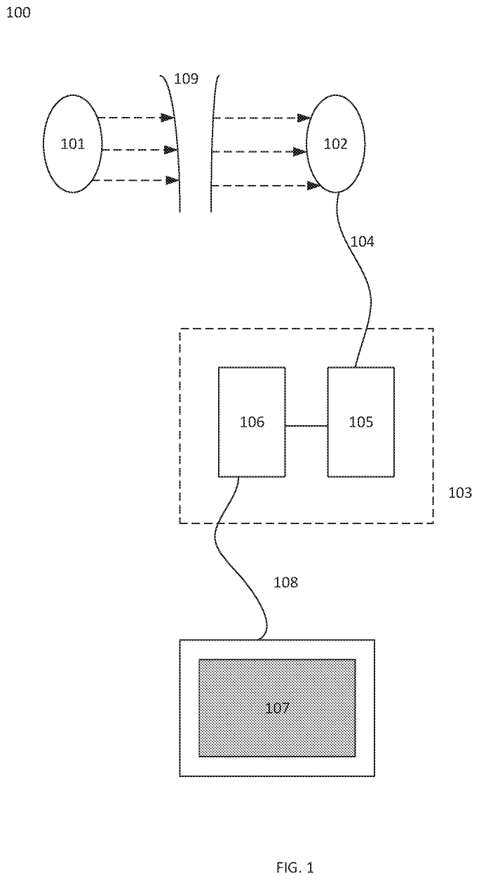

FIG. 1 shows an illustrative embodiment of an integrated apparatus. The integrated apparatus 100 includes a radiation source 101, a detector 102, and a processor 103 that is connected to the detector 102 by a cable 104. The processor includes a video decoder 105 that converts the analog image data from the detector 102 to standard digital video data, which is connected to an enhancement unit 106 that detects one or more straight lines in the video data stream, and incorporates into the video data stream a video representation of a linear extension of the one or more straight lines. The enhancement unit 106 is connected to a monitor 107 using a standard video cable 108. FIG. 1 also shows a portion of the body of a subject 109 undergoing imaging.

In at least another aspect, the disclosure provides methods for imaging, the methods comprising: (a) providing an imaging device; (b) detecting an image using the imagine device, wherein the image is represented by an image data stream; (c) communicating the image data stream to a processor, and, using the processor, (i) converting the image data stream to a video data stream, which provides a video representation of the image, and (ii) detecting one or more straight lines in the video data stream, and incorporating into the video data stream a video representation of a linear extension of the one or more straight lines; and (d) communicating the video data stream to a video display to display a video image based on the video data stream.

The methods disclosed herein include providing an imaging device. Examples of such devices include, but are not limited to, fluoroscopes, CT scanners, ultrasound devices, magnetic resonance imaging (MRI) devices, x-ray devices, endoscopes, elastographs, thermographs, positron emission tomography (PET) devices, and single-photon emission computed tomography (SPECT) devices. In some embodiments, the imagine device is a fluoroscope. In some further embodiments, the fluoroscope includes an x-ray source, a filter, and a detector.

The method also includes detecting an image. The nature of the image will depend, in some ways, on the imaging device. In some embodiments, the image is an image of a portion of the human body, such as an interior portion of the human body that is otherwise not visible by external observation. In some embodiments, the image is an image of a portion of the human body undergoing a surgery or a medical procedure, e.g., that involves inserting one or more medical instruments into a portion of the human body. The detecting includes generating an image data stream by the detector. In most instances, this is an analog signal, although, in some embodiments, it can be a digital signal. The image data stream represents the image recorded by the detector, such as analog image data.

The method also includes communicating the image data stream to a processor, and, using the processor, (i) converting the image data stream to a video data stream, which provides a video representation of the image, and (ii) detecting one or more straight lines in the video data stream, and incorporating into the video data stream a video representation of a linear extension of the one or more straight lines. The image data stream can be communicated to the processor via any suitable wireless or wired means. Various ways of converting the image data stream to a video data stream, such as a standard digital video stream, are described above, which description is incorporated here by reference. Further, various ways of detecting and analyzing one or more straight lines in the video data stream, and incorporating into the video data stream a video representation of a linear extension of the one or more straight lines and information about the one or more straight lines are also described in detail above, which description is also incorporated by reference.

The methods disclosed herein include detecting one or more straight lines in the image data stream. This does not necessarily mean that the processing involved in this detection is carried out directly on the image data stream. In some embodiments, for example, the processor may convert analog image data to a standard digital video format, and then perform the detecting on the data in that standard digital video format.

These one or more straight lines generally represent or correspond to one or more surgical instruments. Examples of such surgical instruments include, but are not limited to, wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof. In that way, the processor is detecting the presence of one or more such surgical instruments within the image that is captured by the detector. This would commonly occur within the context of image-guided surgery or other image-guided medical procedures.

The methods disclosed herein also include communicating the video data stream to a video display to display a video image based on the video data stream. Any suitable video display can be used, including, but not limited to, a monitor, a computer screen, a television screen, and the like. The video data stream can be communicated to the video display in any suitable way, including wired or wireless transmission. In some embodiments, a standard cable having a VGA, DVI, HDMI, or DisplayPort connector can be used.

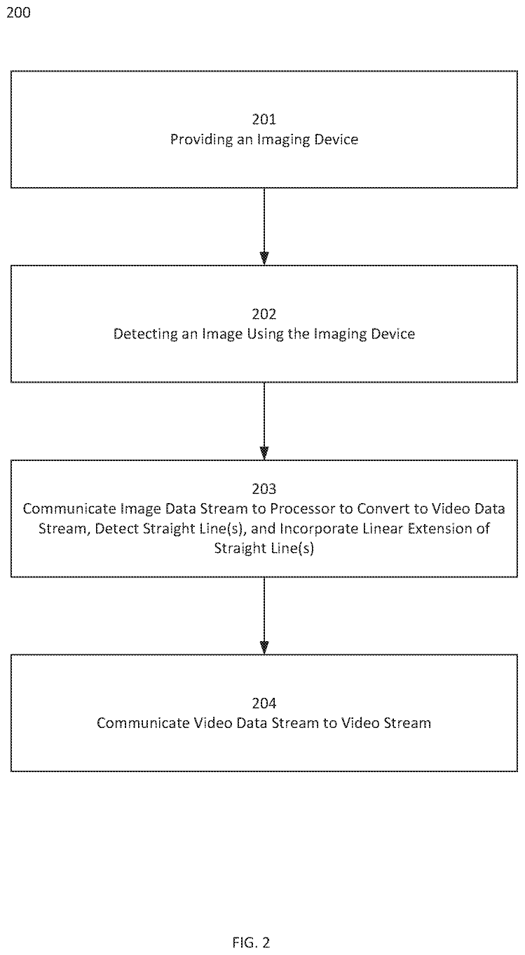

FIG. 2 shows an illustrative embodiment of a method of performing an integrated enhancement of video data. The enhancement method 200 includes: providing an imaging device 201; detecting an image using the imagine device, wherein the image is represented by an image data stream 202; communicating the image data stream to a processor, and, using the processor, (i) converting the image data stream to a video data stream, which provides a video representation of the image, and (ii) detecting one or more straight lines in the video data stream, and incorporating into the video data stream a video representation of a linear extension of the one or more straight lines 203; and communicating the video data stream to a video display to display a video image based on the video data stream 204.

Non-Integrated Video-Enhanced Imaging Apparatus

The aspects set forth above describe apparatuses and methods where the enhancement of the video data stream is carried out in a manner that is integrated into the device. Examples of such devices include devices where such features are incorporated into the hardware or software of the apparatus. In some cases, however, it can also be desirable to perform such enhancement using apparatuses whose hardware or software lack such features.

Thus, in one or more aspects, the disclosure provides apparatuses for enhancing a video data steam, the apparatuses comprising: (a) a video input, which is configured to receive a first video data stream; (b) a processor, which is in electrical communication with the video input, wherein the processor is configured to detect one or more straight lines in the first video data stream, and to convert the first video data stream to a second video data stream, wherein the second video stream comprises a video representation of a linear extension of the one or more straight lines in the first video data stream; and (c) a video display, which is in electrical communication with the processor, and which is configured to display a video image based on the second video data stream.

The apparatuses disclosed herein include a video input. Any suitable device can be used, including wired and wireless devices. In some embodiments, the video input is a plug capable of receiving standard digital video data, such as a cable with a VGA, DVI, HDMI, or DisplayPort interface connector.

The apparatuses disclosed herein also include a processor. Such processors are the same as those described above for the integrated apparatuses, except that they need not include circuitry for converting a non-standard video data stream, such as analog image data stream, to a standard digital video data stream. Even so, the processor nevertheless is configured to detect one or more straight lines in the first video data stream, and to convert the first video data stream to a second video data stream, wherein the second video stream comprises a video representation of a linear extension of the one or more straight lines in the first video data stream.

The apparatuses disclosed herein are configured or programmed to detect one or more straight lines in the image data stream. This does not necessarily mean that the processing involved in this detection is carried out directly on the image data stream. In some embodiments, for example, the processor may convert analog image data to a standard digital video format, and then perform the detecting on the data in that standard digital video format.

These one or more straight lines generally represent or correspond to one or more surgical instruments. Examples of such surgical instruments include, but are not limited to, wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof. In that way, the processor is detecting the presence of one or more such surgical instruments within the image that is captured by the detector. This would commonly occur within the context of image-guided surgery or other image-guided medical procedures.

The detection of the one or more straight lines can be carried out by any suitable algorithm. For example, the detection can be carried out using algorithms based on the Hough transform, and tailored to identify particular features characteristic of the relevant instruments. Examples of these techniques are illustrated in literature relating to the grayscale Hough transform (GSHT), such as Lo et al., Pattern Recognition, vol. 28, pp. 647-661 (1994), which is incorporated herein by reference. The manipulation of the original image is typically required before a line detection algorithm is applied. This manipulation may include converting a color image to a grayscale image, isolating a specific region of interest, smoothing to remove edges caused by artifacts, converting to a binary image, reducing the width of large objects. Once the image has been manipulated, the Hough transform implements a voting scheme for determining the presence and location of a line within the image. Possible lines must receive a user specified number of votes to be considered detected in the image. In some embodiments, the pixels that are considered important by the binary filter each vote for the set of lines they could lie on. After iterating through all the pixels, a threshold must be reached to be considered a line.

The apparatuses disclosed herein are also configured to incorporate into the video data stream a video representation of a linear extension of the one or more straight lines in the image data stream. In some embodiments, this occurs following the above-described straight-line detection. Determining the tip or end of the device can be used to determine the direction that the linear extension should be performed. The linear extension is drawn along the detected line from the tip of the device to the edge of the image in the direction moving away from the device. The Bresenham's line algorithm is used for generating the path of the linear guide extension given two points and user defined thickness. This generated linear guide extension is then merged with the original image which may completely or partially cover the portions of the original image with the linear guide. For example, in some instances, There is a user defined transparency of combining these two images together. Thus, a user can see both what is underneath the original image and the generated guidance.

In some embodiments, the apparatuses disclosed herein include a processor that is further configured to determine a direction of motion of any of the one or more straight lines in the image data stream. To recognize the same line in a series of image, an estimation of a line's movement is required. This can be achieved by an assumption if a line in the previous image and a line in the current image have similar position and orientation, they are the same line. The speed of movement of this line can be determined and predicted for the proceeding images.

In some further embodiments, the apparatuses disclosed herein include a feature where the video data stream further comprises a video representation of an error bar. In some embodiments, the error bar corresponds to a confidence interval in calculating the linear extension of the one or more straight lines in the image data stream. In some embodiments, the confidence interval is a 80% confidence interval, or a 90% confidence interval, or a 95% confidence interval, or a 99% confidence interval. The confidence interval is a calculated value representative of the deviation from the predicted location and orientation of a line to the line detected in the current image. The calculated value can be dependent on the speed of the line to achieve an accurate confidence value for slower and faster moving line.

In some embodiments, the video data stream further comprises a video representation of a number, wherein the number is a text representation of the number of straight lines detected in the image data stream. The system can count the number of lines detected. This number is then overlaid onto the user display image as text. This text can be generated using one of the many font libraries available which, given the text content and location, will overlay the text onto the user display image.

In some embodiments, the video data stream further comprises a video representation of an indicator, wherein the indicator appears on the final user display image at each point two or more of the detected straight lines intersect. Line-line intersections in a plane can be determined using the line equation. When lines are detected, line equations are obtained. From these equations, the intersection points of all detected lines can be calculated based on Euclidian geometry. At the location of each intersection point, a font library can be used to generate a symbol that is then overlaid onto the user display image as an intersection indicator.

In some embodiments, the processor is further configured to determine that bone material is near a detected line. When the system determines that bone material, both cortical and trabecular, is near a detected line, the system will overlay the bone material region with a transparent color or outline. An embodiment of the system may use some or all of the available information to determine the presence and location of bone within the image: intensity of the pixels, the general shape of pixels, the relative location of pixels, and input generated by the user. Having determined the area of bone material within an image, the perimeter of this area can be divided into specific points. Given each point of the perimeter and each point of the previously detected line, the distance can be calculated. If the distance is shorter than a user specified amount, the bone region is then marked to be highlighted. The portion of the bone to be highlighted can be merged with a given color to produce a transparent image that is then shown on the user display image.

In some embodiments, the processor is further configured to determine a centroid of a region of detected bone, wherein all portions of the region of detected bone are weighed equally in the determination. Determining the centroid of a given region of bone can be performed by calculations of image intensity moments. For the bone area, the intensity is assumed to be constant throughout the entire region. From these moment values, the centroid is at the intersection of two neutral axis which can be calculated from the first moment. In some embodiments, the video data stream further comprises an indicator at each centroid of a region of detected bone. At each determined centroid, a font library can be used to generate a symbol that is then overlaid onto the user display image as a centroid indicator.

In some embodiments, the processor is further configured to determine a centroid of a region of detected bone, wherein portions of the region of detected bone are weighed based on their intensity in the user data stream in the determination. Determining the centroid of a given region of bone can be performed by calculations of image intensity moments. Due to the non-uniformity of bone density, the centroid calculations can use the intensity of the original image to correlate with density. From these moment values, the centroid is at the intersection of two neutral axis which can be calculated from the first moment. In some embodiments, the video data stream further comprises an indicator at each centroid of a region of detected bone. At each determined centroid, a font library can be used to generate a symbol that is then overlaid onto the user display image as a centroid indicator.

In some embodiments, the video data stream further comprises a video representation identifying at least one individual line, wherein the at least one line is identified by a unique label on the final user display image. The same line can be identified with a specific text label or color throughout a series of continuous images. Forming an estimation of the line's motion can enable the prediction of a line in the consecutive image. If a line is within a tolerance range of this predicted line, then it will be considered the same line. The same color or label for this line can be applied to the original image so said unique identifier appears on the user display image.

In some embodiments, the processor detected and indicated placed screw trajectories using labels, colors, or other unique indicators. Using the same instrument detection, trajectory projection, and bone detection algorithms described previously, screws may be individually identified and labeled. Their cross section may be overlaid with a transparent color or outline using the following: intensity of the pixels, general shape of pixels, relative location of pixels, and user-generated input. The screw trajectory can be displayed as a short line segment or as a continuous line that runs through the entire user display image. This line may be used in the calculation of intersections and angles with other trajectory paths.

In some embodiments, once the processor has identified line representations from at least a pair of devices, the angle between the two lines can be calculated and displayed on the final user display image. The location of this angle may or may not be placed near the point of intersection. Additionally, a curved line may be drawn connecting the two intersecting lines. The region bordered by the two lines and the curved line may be shaded. The line equations can be determined from the line detection. The equation for two lines can then be used to directly find the angle between the two lines based on Euclidian geometry. The angle value can be placed near the intersection point using a text label from a standard font library.

The apparatuses disclosed herein also include a video display, which is in electrical communication with the processor, and which is configured to display a video image based on the video data stream. Any suitable video display can be used, including, but not limited to, a monitor, a computer screen, a television screen, and the like. The video data stream can be communicated to the video display in any suitable way, including wired or wireless transmission. In some embodiments, a standard cable having a VGA, DVI, HDMI, or DisplayPort connector can be used.

The apparatuses disclosed herein, in some embodiments, represent an image detected by a medical imaging device, such as a medical imaging device that at least includes a radiation source and a detector. Examples of such devices include, but are not limited to, fluoroscopes, CT scanners, ultrasound devices, magnetic resonance imaging (MRI) devices, x-ray devices, endoscopes, elastographs, thermographs, positron emission tomography (PET) devices, and single-photon emission computed tomography (SPECT) devices. In some embodiments, the imagine device is a fluoroscope. In some further embodiments, the fluoroscope includes an x-ray source, a filter, and a detector.

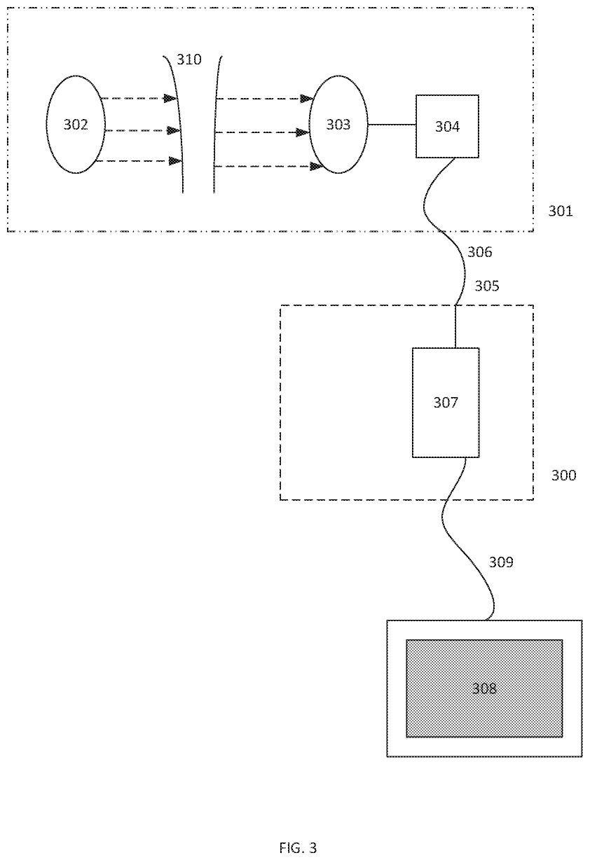

FIG. 3 shows an illustrative embodiment of a non-integrated apparatus. The non-integrated enhancement apparatus 300 may operate in conjunction with an imaging device 301 having a radiation source 302 and a detector 303, and a processor 304 that generates a standard digital video data. The imaging device 301 is connected to the video input 305 of the enhancement apparatus 300 via a video cable 306, such that the standard digital video output of the imaging device 301 can be communicated to the enhancement device 300. The enhancement device 300 contains a processor that includes an enhancement unit 307 that detects one or more straight lines in the video data stream from the imaging device 301, and converts the that video data stream to a second video data stream, wherein the second video stream includes a video representation of a linear extension of the one or more straight lines in the detected in the original video data stream. The enhancement unit 307 is connected to a monitor 308 using a standard video cable 309. FIG. 3 also shows a portion of the body of a subject 310 undergoing imaging.

In at least another aspect, the disclosure provides methods of enhancing a video data stream, the methods comprising: (a) providing a first video data stream, which represents a video image; (b) communicating the first video data stream to a processor, and, using the processor, detecting one or more straight lines in the image, and converting the first video data stream to a second video data stream, wherein the second video data stream comprises a video representation of a linear extension of the one or more straight lines in the image; and (c) communicating the second video data stream to a video display to display a video image based on the second video data stream.

The methods disclosed herein include providing a first video data stream, which represents a video image. In some embodiments, the video data stream is in a standard digital video format. In some embodiments, the first video data stream is the standard digital video data stream generated by a medical imaging device for display. In such embodiments, the video data stream is diverted and enhanced prior to its display on a display device, such as a monitor. Examples of such devices include, but are not limited to, fluoroscopes, CT scanners, ultrasound devices, magnetic resonance imaging (MRI) devices, x-ray devices, endoscopes, elastographs, thermographs, positron emission tomography (PET) devices, and single-photon emission computed tomography (SPECT) devices. In some embodiments, the imagine device is a fluoroscope. In some further embodiments, the fluoroscope includes an x-ray source, a filter, and a detector.

The method also includes communicating the image data stream to a processor, and, using the processor, detecting one or more straight lines in the image, and converting the first video data stream to a second video data stream, wherein the second video data stream comprises a video representation of a linear extension of the one or more straight lines in the image. Further, various ways of detecting and analyzing one or more straight lines in a video data stream, and incorporating into the video data stream a video representation of a linear extension of the one or more straight lines and information about the one or more straight lines are also described in detail above, which description is also incorporated by reference.

These one or more straight lines generally represent or correspond to one or more surgical instruments. Examples of such surgical instruments include, but are not limited to, wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof. In that way, the processor is detecting the presence of one or more such surgical instruments within the image that is captured by the detector. This would commonly occur within the context of image-guided surgery or other image-guided medical procedures.

The methods disclosed herein also include communicating the video data stream to a video display to display a video image based on the video data stream. Any suitable video display can be used, including, but not limited to, a monitor, a computer screen, a television screen, and the like. The video data stream can be communicated to the video display in any suitable way, including wired or wireless transmission. In some embodiments, a standard cable having a VGA, DVI, HDMI, or DisplayPort connector can be used.

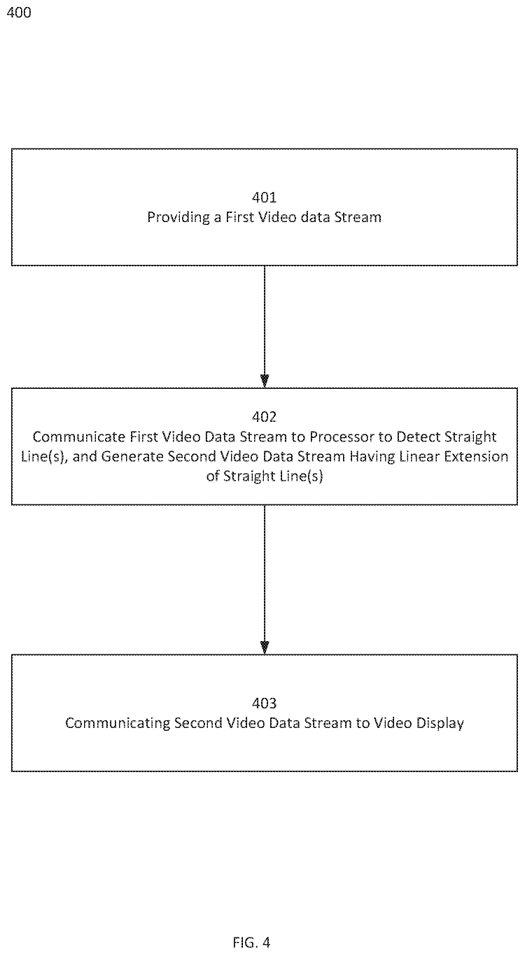

FIG. 4 shows an illustrative embodiment of a method of performing a non-integrated enhancement of video data. The enhancement method 400 includes: providing a first video data stream, which represents a video image 401; communicating the first video data stream to a processor, and, using the processor, detecting one or more straight lines in the image, and converting the first video data stream to a second video data stream, wherein the second video data stream comprises a video representation of a linear extension of the one or more straight lines in the image 402; and communicating the second video data stream to a video display to display a video image based on the second video data stream 403.

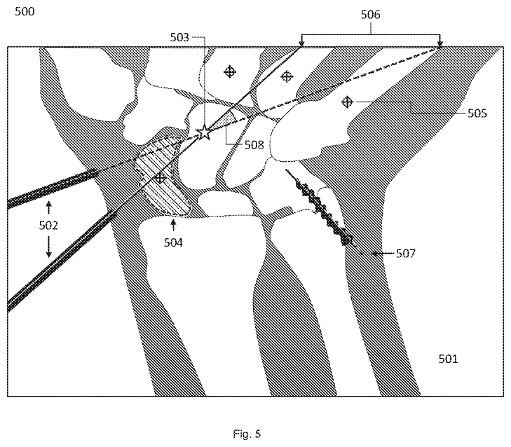

FIG. 5 shows an illustrative embodiment of a final user display image. The final user display image 500 shows a portion of the body 501 of a subject undergoing imaging. The final user display image 500 also shows the one or more straight lines detected in the video data stream 502 that correspond to one or more surgical instruments. In some embodiments, an intersection indicator 503 appears on the final user display image 500 at the point two or more detected straight lines will intersect. In some embodiments, when the system determines that bone material is near a detected line 502, the system will overlay the bone material region with shading, a color, and/or an outline 504 on the final user display image 500. In some embodiments, a centroid indicator 505 appears on the final user display image 500 at the point determined to be a centroid of a region of detected bone. In some embodiments, each extension 506 of each detected line 502 has a unique color, line type, and/or line weight to differentiate each line extension on the final user display image 500. In some embodiments, when screws are detected, the screw trajectory 507 can be displayed as a short line segment on the final user display image 500. In some embodiments, the angle between two detected straight lines can be indicated by a curved line connecting the two intersecting lines and the region between the two lines and the curve shaded 508 on the final user display image 500.

### EXAMPLES

### Example 1—Embodiments

Embodiments of the present disclosure include:
A₁. An apparatus for medical imaging, the apparatus comprising:

(a) an imaging device, which comprises a radiation source and a detector;

(b) a processor, which is in electrical communication with the detector, and which is configured to receive an image data stream from the detector;
(i) wherein the processor is configured to convert the image data stream to a video data stream, which provides a video representation of the image data stream; and
(ii) wherein the processor is further configured to detect one or more straight lines in the image data stream, and to incorporate into the video data stream a video representation of a linear extension of the one or more straight lines in the image data stream; and

(c) a video display, which is in electrical communication with the processor, and which is configured to display a video image based on the video data stream.

A2. The apparatus of embodiment A1, wherein the processor comprises a video capture card.
A3. The apparatus of embodiments A1 or A2, wherein the imaging device is a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.
A4. The apparatus of embodiment A3, wherein the imaging device is a fluoroscope.
A5. The apparatus of any one of embodiments A1 to A4, wherein the processor is comprised by a computer.
A6. The apparatus of any one of embodiments A1 to A5, wherein the one or more straight lines represent one or more surgical instruments.
A7. The apparatus of embodiment A6, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.
A8. The apparatus of any one of embodiments A1 to A7, wherein the processor is further configured to determine a direction of motion of any of the one or more straight lines in the image data stream.
A9. The apparatus of any one of embodiments A1 to A8, wherein the video data stream further comprises a video representation of an error bar, which corresponds to a confidence interval in calculating the linear extension of the one or more straight lines in the image data stream.
A10. The apparatus of any of embodiments A1 to A9, wherein the video data stream further comprises a video representation of a number, wherein the number is a text representation of the number of straight lines detected in the image data stream.
A11. The apparatus of any of embodiments A1 to A10, wherein the video data stream further comprises a video representation of an indicator, wherein the indicator appears on the final user display image at each point two or more of the detected straight lines intersect.
A12. The apparatus of any of embodiments A1 to A11, wherein the processor is further configured to determine that bone material is near a detected line
A13. The apparatus of any of embodiments A1 to A12, wherein the processor is further configured to determine a centroid of a region of detected bone, wherein all portions of the region of detected bone are weighed equally in the determination.
A14. The apparatus of any of embodiments A1 to A12, wherein the processor is further configured to determine a centroid of a region of detected bone, wherein portions of the region of detected bone are weighed based on their intensity in the image data stream.
A15. The apparatus of any of embodiments A1 to A14, wherein the video data stream further comprises a video representation identifying at least one individual line, wherein the at least one line is identified by a unique label on the final user display image.
A16. The apparatus of embodiment A15, wherein at least one uniquely labeled individual line represents a placed screw.
A17. The apparatus of any of embodiments A1 to A16, wherein if at least two individual lines are detected in the image data stream, the video data stream further comprises a video representation of the angle at the intersection point of the at least two lines.
A18. The apparatus of any one of embodiments A1 to A17, wherein the video display is a local monitor.
B1. A method for imaging, the method comprising:

(a) providing an imaging device;

(b) detecting an image using the imagine device, wherein the image is represented by an image data stream;

(c) communicating the image data stream to a processor, and, using the processor, (i) converting the image data stream to a video data stream, which provides a video representation of the image, and (ii) detecting one or more straight lines in the image, and incorporating into the video data stream a video representation of a linear extension of the one or more straight lines in the image; and

(d) communicating the video data stream to a video display to display a video image based on the video data stream.

B2. The method of embodiment B1, wherein the processor comprises a video capture card.
B3. The method of embodiments B1 or B2, wherein the imaging device is a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.
B4. The method of embodiment B3, wherein the imaging device is a fluoroscope.
B5. The method of any one of embodiments B1 to B4, wherein the processor is comprised by the computer.
B6. The method of any one of embodiments B1 to B5, wherein the one or more straight lines represent one or more surgical instruments.
B7. The method of embodiment B6, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.
B8. The method of any one of embodiments B1 to B7, wherein the processor determines a direction of motion of any of the one or more straight lines in the image data stream.
B9. The method of any one of embodiments B1 to B8, wherein the video data stream further comprises a video representation of an error bar, which corresponds to a confidence interval in calculating the linear extension of the one or more straight lines in the image data stream.
B10. The method of any one of embodiments B1 to B9, wherein the video display is a local monitor.
B11. The method of any one of embodiments B1 to B10, wherein the video image represents a surgical procedure.
C1. An apparatus for enhancing a video data steam, the apparatus comprising:

(a) a video input, which is configured to receive a first video data stream;

(b) a processor, which is in electrical communication with the video input, wherein the processor is configured to detect one or more straight lines in the first video data stream, and to convert the first video data stream to a second video data stream, wherein the second video stream comprises a video representation of a linear extension of the one or more straight lines in the first video data stream; and

(c) a video display, which is in electrical communication with the processor, and which is configured to display a video image based on the second video data stream.

C2. The apparatus of embodiment C1, wherein the processor comprises a video capture card.
C3. The apparatus of embodiments C1 or C2, wherein the first video data stream represents image data detected by an imaging device, such as a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.
C4. The apparatus of embodiment C3, wherein the imaging device is a fluoroscope.
C5. The apparatus of any one of embodiments C1 to C4, wherein the processor is comprised by a computer.
C6. The apparatus of any one of embodiments C1 to C5, wherein the one or more straight lines represent one or more surgical instruments.
C7. The apparatus of embodiment C6, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.
C8. The apparatus of any one of embodiments C1 to C7, wherein the processor is further configured to determine a direction of motion of any of the one or more straight lines in the first video data stream.
C9. The apparatus of any one of embodiments C1 to C8, wherein the second video data stream further comprises a video representation of an error bar, which corresponds to a confidence interval in calculating the linear extension of the one or more straight lines in the first video data stream.
C10. The apparatus of any of embodiments C1 to C9, wherein the video data stream further comprises a video representation of a number, wherein the number is a text representation of the number of straight lines detected in the image data stream.
C11. The apparatus of any of embodiments C1 to C10, wherein the video data stream further comprises a video representation of an indicator, wherein the indicator appears on the final user display image at each point two or more of the detected straight lines intersect.
C12. The apparatus of any of embodiments C1 to C11, wherein the processor is further configured to determine that bone material is near a detected line
C13. The apparatus of any of embodiments C1 to C12, wherein the processor is further configured to determine a centroid of a region of detected bone, wherein all portions of the region of detected bone are weighed equally in the determination.
C14. The apparatus of any of embodiments C1 to C13, wherein the processor is further configured to determine a centroid of a region of detected bone, wherein portions of the region of detected bone are weighed based on their intensity in the image data stream.
C15. The apparatus of any of embodiments C1 to C14, wherein the video data stream further comprises a video representation identifying at least one individual line, wherein the at least one line is identified by a unique label on the final user display image.
C16. The apparatus of embodiment C15, wherein at least one uniquely labeled individual line represents a placed screw.
C17. The apparatus of any of embodiments C1 to C16, wherein if at least two individual lines are detected in the image data stream, the video data stream further comprises a video representation of the angle at the intersection point of the at least two lines.
C18. The apparatus of any one of embodiments C1 to C17, wherein the video display is a local monitor.
D1. A method of enhancing a video data stream, the method comprising:

(a) providing a first video data stream, which represents a video image;

(b) communicating the first video data stream to a processor, and, using the processor, detecting one or more straight lines in the image, and converting the first video data stream to a second video data stream, wherein the second video data stream comprises a video representation of a linear extension of the one or more straight lines in the image; and

(c) communicating the second video data stream to a video display to display a video image based on the second video data stream.

D2. The method of embodiment D1, wherein the processor comprises a video capture card.
D3. The method of embodiments D1 or D2, wherein the image is detected by an imaging device, such as a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.
D4. The method of embodiment D3, wherein the imaging device is a fluoroscope.
D5. The method of any one of embodiments D1 to D4, wherein the processor is comprised by the computer.
D6. The method of any one of embodiments D1 to D5, wherein the one or more straight lines represent one or more surgical instruments.
D7. The method of embodiment D6, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.
D8. The method of any one of embodiments D1 to D7, wherein the processor determines a direction of motion of any of the one or more straight lines in the first video data stream.
D9. The method of any one of embodiments D1 to D8, wherein the second video data stream further comprises a video representation of an error bar, which corresponds to a confidence interval in calculating the linear extension of the one or more straight lines in the first video data stream.
D10. The method of any one of embodiments D1 to D9, wherein the video display is a local monitor.
D11. The method of any one of embodiments D1 to D10, wherein the video image represents a surgical procedure.

1. An apparatus for medical imaging, the apparatus comprising:
(a) an imaging device, which comprises a radiation source and a detector;
(b) a processor, which is in electrical communication with the detector, and which is configured to receive an image data stream from the detector;
(i) wherein the processor is configured to convert the image data stream to a video data stream, which provides a video representation of the image data stream; and
(ii) wherein the processor is further configured to detect one or more straight lines in the image data stream, and to incorporate into the video data stream a video representation of a linear extension of the one or more straight lines in the image data stream; and
(c) a video display, which is in electrical communication with the processor, and which is configured to display a video image based on the video data stream.

2. The apparatus of claim 1, wherein the processor comprises a video capture card.

3. The apparatus of claim 1, wherein the imaging device is a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.

4. The apparatus of claim 1, wherein the processor is comprised by a computer.

5. The apparatus of claim 1, wherein the one or more straight lines represent one or more surgical instruments, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.

6. The apparatus of claim 1, wherein the processor is further configured to determine a direction of motion of any of the one or more straight lines in the image data stream.

7. The apparatus of claim 1, wherein the video data stream further comprises a video representation of an error bar, which corresponds to a confidence interval in calculating the linear extension of the one or more straight lines in the image data stream.

8. The apparatus of claim 1, wherein the video data stream further comprises a video representation of a number, wherein the number is a text representation of the number of straight lines detected in the image data stream.

9. The apparatus of claim 1, wherein the video data stream further comprises a video representation of an indicator, wherein the indicator appears on the final user display image at each point two or more of the detected straight lines intersect.

10. The apparatus of claim 1, wherein the processor is configured to detect bone material.

11. The apparatus of claim 10, wherein the processor is further configured to determine a centroid of a region of detected bone, wherein all portions of the region of detected bone are weighed equally in the determination.

12. The apparatus of claim 10, wherein the processor is further configured to determine a centroid of a region of detected bone, wherein portions of the region of detected bone are weighed based on their intensity in the image data stream.

13. The apparatus of claim 1, wherein the video data stream further comprises a video representation identifying at least one individual line, wherein the at least one line is identified by a unique label on the final user display image.

14. The apparatus of claim 13, wherein at least one uniquely labeled individual line represents a placed screw.

15. The apparatus of claim 1, wherein if at least two individual lines are detected in the image data stream, the video data stream further comprises a video representation of the angle at the intersection point of the at least two lines.

16. A method for imaging, the method comprising:
(a) providing an imaging device;
(b) detecting an image using the imagine device, wherein the image is represented by an image data stream;
(c) communicating the image data stream to a processor, and, using the processor, (i) converting the image data stream to a video data stream, which provides a video representation of the image, and (ii) detecting one or more straight lines in the image, and incorporating into the video data stream a video representation of a linear extension of the one or more straight lines in the image; and
(d) communicating the video data stream to a video display to display a video image based on the video data stream.

17. The method of claim 16, wherein the imaging device is a fluoroscope, a CT scanner, an ultrasound, a magnetic resonance imaging (MRI) device, an x-ray device, an endoscope, an elastograph, a thermograph, a positron emission tomography (PET) device, or a single-photon emission computed tomography (SPECT) device.

18. The method of claim 16, wherein the one or more straight lines represent one or more surgical instruments, wherein the one or more surgical instruments comprise wires, pins, needles, scalpels, retractors, lancets, drill bits, screws, trocars, ligasures, dilators, speculas, tubes, tips, sealing devices, scopes, probes, endoscopes, carriers, applicators, laser guides, or any combination thereof.

19. The method of claim 16, wherein the processor determines a direction of motion of any of the one or more straight lines in the image data stream.

20. The method of claim 16, wherein the video data stream further comprises a video representation of an error bar, which corresponds to a confidence interval in calculating the linear extension of the one or more straight lines in the image data stream.
